(19) United States
(12) Patent Application Publication
(10) Pub. No.: US 2026/0034153 A1
(43) Pub. Date: Feb. 5, 2026

(54) Cerebral Blood Flow Augmenter, Brain Activity Promoter, and Vigilance Improver

(71) Applicant: Mitsui DM Sugar Co., Ltd., Tokyo (JP)

(72) Inventors: Miki Sakazaki, Shinjuku-ku, Tokyo (JP); Natsumi Kusumi Yamamoto, Shinjuku-ku, Tokyo (JP); Yukie Nagai, Shinjuku-ku, Tokyo (JP)

(73) Assignee: Mitsui DM Sugar Co., Ltd., Tokyo (JP)

(21) Appl. No.: 19/100,177

(22) PCT Filed: Aug. 4, 2023
(86) PCT No.: PCT/JP2023/028565

§ 371 Date: Jan. 31, 2025

(30) Foreign Application Priority Data
Aug. 4, 2022 (JP) 2022-124891

(51) Int. Cl. A61K 31/7016; A61P 9/00; A61P 25/00; A61P 25/28

(52) U.S. Cl. CPC A61K 31/7016; A61P 9/00; A61P 25/00; A61P 25/28

(57) ABSTRACT

The present invention relates to a cerebral blood flow augmenter containing isomaltulose as an active ingredient.

### TECHNICAL FIELD

The present invention relates to a cerebral blood flow augmenter, a brain activity promoter, and a vigilance improver.

### BACKGROUND ART

Isomaltulose is a compound, which is also called as 6-O-α-D-glucopyranosyl-D-fructose (PALATINOSE as a trademark), is known as a carbohydrate having various functions. For example, Patent Literature 1 discloses that diabetic nephropathy is suppressed from worsening by intake of isomaltulose.

### CITATION LIST

### Patent Literature

Patent Literature 1: Japanese Unexamined Patent Publication No. 2016-41661

### Non Patent Literature

Non Patent Literature 1: Joel S. Warm et al., Vigilance requires hard mental work and is stressful, Human Factors, Vol. 50 (3), pp. 433-41, 2008.
Non Patent Literature 2: Watson, et al., The impact of blackcurrant juice on attention, mood and brain wave spectral activity in young healthy volunteers, Nutritional Neuroscience, Vol. 22 (8), pp. 596-606, 2019.

### SUMMARY OF INVENTION

### Technical Problem

An object of the present invention is to provide a novel use of isomaltulose for further effectively using isomaltulose.

### Solution to Problem

The present inventors found that isomaltulose has a cerebral blood flow increasing action, a brain activity promoting action, and a vigilance-level improving action. The present invention is based on these findings.

As an aspect of the present invention, there is provided a cerebral blood flow augmenter, a brain activity promoter, or a vigilance improver containing isomaltulose as an active ingredient. Since isomaltulose has a cerebral blood flow increasing action, a brain activity promoting action, and a vigilance-level improving action, as described above, the above agent can increase the cerebral blood flow, thereby promoting the brain activity and improving the vigilance.

The present invention can also be regarded as a method for increasing cerebral blood flow of a subject, including a step of administering an effective amount of a cerebral blood flow augmenter containing isomaltulose as an active ingredient to the subject in need thereof. The present invention can also be regarded as a method for promoting brain activity of a subject, including a step of administering an effective amount of a brain activity promoter containing isomaltulose as an active ingredient to the subject in need thereof. The present invention can also be regarded as a method for improving the vigilance of a subject, including a step of administering an effective amount of a vigilance improver containing isomaltulose as an active ingredient to the subject in need thereof.

The present invention can also be regarded as use of isomaltulose for producing a cerebral blood flow augmenter. The present invention can also be regarded as use of isomaltulose for producing a brain activity promoter. The present invention can further be regarded as use of isomaltulose for producing a vigilance improver.

The present invention can also be regarded as isomaltulose for use in increasing cerebral blood flow. The present invention can also be regarded as isomaltulose for use in promoting brain activity. The present invention can also be regarded as isomaltulose for use in improving the vigilance.

### Advantageous Effects of Invention

According to the present invention, a novel use of isomaltulose is provided.

### BRIEF DESCRIPTION OF DRAWINGS

FIG. 1 is a graph showing a transition of response time of a subject during the implementation of DVT. Comparison between groups was performed using Bonferroni's multiple comparison test.

FIG. 2 is a graph showing the transition of an average value of a cerebral blood flow change over time, with the cerebral blood flow at the start of DVT set to zero.

FIG. 3 is a graph showing an average value of the cerebral blood flow change from of 0 to 120 seconds elapsed time, based on the transition of the average value of cerebral blood flow change shown in FIG. 2. Comparison between groups was performed using unpaired t-test.

### DESCRIPTION OF EMBODIMENTS

Hereinafter, embodiments for carrying out the present invention will be described in detail. However, the present invention is not limited to the following embodiments.

The cerebral blood flow augmenter according to the present embodiment contains isomaltulose as an active ingredient.

Isomaltulose is a compound produced by binding glucose and fructose via α-1,6 bond, and also called as 6-O-α-D-glucopyranosyl-D-fructose. Isomaltulose is also called as PALATINOSE. Note that “PALATINOSE” is a registered trademark of Mitsui DM Sugar Co., Ltd.

Isomaltulose is found in honey, in nature. Isomaltulose is also present in a product obtained by transferring a sugar moiety when α-glucosyltransferase (isomaltulose synthase) derived from bacteria or yeast acts on sucrose. Industrially, isomaltulose is produced by allowing an α-glucosyltransferase derived from a bacterium such as Protaminobacter rubrum or Serratia plymuthica to act on sucrose.

As the isomaltulose to be used for the cerebral blood flow augmenter, a naturally occurring isomaltulose or an isomaltulose synthesized by, e.g., an enzymatic action, may be used.

Isomaltulose may be contained in the cerebral blood flow augmenter as crystal particles, or granular particles. The granular particles contains, for example, (spherical) particles containing an aggregate of isomaltulose crystal particles and amorphous sugar, in which the sugar may be encased within the aggregate of crystal particles. Such granular particles can be obtained, for example, by precipitating isomaltulose crystal particles from a carbohydrate solution containing isomaltulose and an amorphous sugar, and spray-drying the carbohydrate solution containing the crystal particles. Alternatively, granular particles containing isomaltulose can be obtained by applying shear force to the carbohydrate solution while heating to allow isomaltulose crystal nuclei to precipitate and cooling a mixture containing the crystal nuclei. The carbohydrate solution can be obtained, for example, by allowing an enzyme to act on sucrose. In this case, the carbohydrate solution and the granular particles to be obtained contain, e.g., trehalose, fructose, glucose, sucrose, and isomaltose as amorphous sugars. The granular particles may be a solid described, for example, in Japanese Unexamined Patent Publication No. 2012-179045.

Also, isomaltulose may be a commercially available product. Examples of the commercially available product include crystalline PALATINOSE (trade name: “CRYSTALLINE PALATINOSE PST-N”, Mitsui DM Sugar Co., Ltd.), powdered PALATINOSE (trade name: “POWDERED PALATINOSE PST-NP”, Mitsui DM Sugar Co., Ltd.), and PALATINOSE syrup (trade name: “PARATINOUS SYRUP-ISN” and “PARATINOUS SYRUP-TN”, Mitsui DM Sugar Co., Ltd.).

The cerebral blood flow augmenter according to the present embodiment has an action of increasing cerebral blood flow (blood flow in the brain). The phase “Increasing cerebral blood flow” may be to increase blood flow at a site in the brain, for example, to increase blood flow in the dorsal lateral prefrontal area responsible for, e.g., learning, inference, and suppression. The action of increasing cerebral blood flow herein includes an action of improving cerebral blood flow (an action of recovering from a decreased cerebral blood flow) and an action of suppressing cerebral blood flow from decreasing.

Cerebral blood flow can be measured based on near-Infrared spectroscopy (fNIRS), which is a non-invasive optical imaging technique for monitoring the oxygen state of tissues. Since fNIRS is a method of checking a change in cerebral blood flow by irradiating the head with imperceptible near-infrared rays (650 to 1000 nm), which is also contained in sunlight, measurement can be performed highly safe, and the size of the apparatus can be reduced. The measurement of the cerebral blood flow using fNIRS can be performed, for example, by a portable brain activity measuring apparatus (HOT-2000) manufactured by NeU Corporation.

The cerebral blood flow augmenter of the present embodiment has an action of promoting brain activity based on the action of increasing cerebral blood flow. The action of promoting brain activity in the specification includes an action of improving brain activity (an action of recovering from decreased brain activity) and an action of suppressing brain activity from decreasing.

The cerebral blood flow augmenter of the present embodiment has an action of improving the vigilance based on the action of increasing the cerebral blood flow. The vigilance refers to the ability to maintain attention and alertness for a long time. The action of improving the vigilance in the specification includes an action of improving the vigilance (an action of recovering from a decreased vigilance), an action of suppressing the vigilance from decreasing, and an action of maintaining a state of a high vigilance. In addition, the action of improving the vigilance in the specification also includes an action of sustaining a state in which processing time, response time, or reaction time for a given task, such as a cognitive task, is fast for an extended period.

The cerebral blood flow augmenter of the present embodiment also has an action of improving headache, an action of improving fatigue, an action of improving irritation, an action of maintaining concentration, an action of maintaining ability to study or do office work, an action of improving strategy or physical function in sports, and an action of maintaining motivation to work, based on the action of increasing cerebral blood flow.

The cerebral blood flow augmenter according to the present embodiment contains isomaltulose as an active ingredient. The cerebral blood flow augmenter may be constituted of isomaltulose alone, or a composition containing isomaltulose. When the cerebral blood flow augmenter is a composition, isomaltulose to be contained in the composition may be a single substance or a commercially available isomaltulose preparation containing several saccharides. The isomaltulose preparation may contain components other than isomaltulose.

The other components may be materials that can be used in foods, quasi-drugs, or pharmaceuticals. Examples of the materials that can be used for foods, quasi-pharmaceutical products, or pharmaceuticals include, but are not particularly limited to, amino acids, proteins, carbohydrates, fats and oils, sweeteners, minerals, vitamins, flavors, excipients, binders, lubricants, disintegrants, emulsifiers, surfactants, bases, solubilizing agents and suspending agents.

Examples of the proteins include milk casein, whey, soybean protein, wheat protein, and albumen. Examples of the carbohydrates include corn starch, cellulose, pregelatinized starch, wheat starch, rice starch, and potato starch. Examples of the fats and oils include salad oil, corn oil, soybean oil, safflower oil, olive oil, and palm oil. Examples of the sweeteners include saccharides such as glucose, sucrose, fructose, and high-fructose corn syrup, sugar alcohols such as xylitol, erythritol, and maltitol, artificial sweeteners such as sucralose, aspartame, saccharin, and acesulfame K, and stevia sweeteners. Examples of the minerals include calcium, potassium, phosphorus, sodium, manganese, iron, zinc, magnesium, and salts of these. Examples of the vitamins include vitamin E, vitamin C, vitamin A, vitamin D, vitamin B, biotin, and niacin. Examples of the excipients include dextrin, starch, lactose, and crystalline cellulose. Examples of the binders include polyvinyl alcohol, gelatin, hydroxypropyl methyl cellulose, hydroxypropyl cellulose, sodium carboxymethyl cellulose, and polyvinylpyrrolidone. Examples of the lubricants include magnesium stearate, calcium stearate, and talc. Examples of the disintegrants include crystalline cellulose, agar, gelatin, calcium carbonate, sodium hydrogen carbonate, and dextrin. Examples of the emulsifiers or surfactants include sucrose fatty acid ester, citric acid, lactic acid, glycerin fatty acid ester, polyglyceryl fatty acid ester, sorbitan fatty acid ester, propylene glycol fatty acid ester, and lecithin. Examples of the bases include cetostearyl alcohol, lanolin, and polyethylene glycol. Examples of the solubilizing agents include polyethylene glycol, propylene glycol, sodium carbonate, and sodium citrate. Examples of the suspending agents include glyceryl monostearate, polyvinyl alcohol, polyvinyl pyrrolidone, methyl cellulose, hydroxymethyl cellulose, and sodium alginate. These may be used singly or in combination of two or more types thereof.

Since isomaltulose has about ½ as sweet as sucrose, the cerebral blood flow augmenter according to the present embodiment may contain isomaltulose principally as a sweetener. Isomaltulose has ⅕ as low as hydrolysis rate of sucrose, and can suppress blood glucose level from increasing after intake. Thus, due to the content of isomaltulose principally as a sweetener, it is possible to obtain also an effect of easily controlling blood glucose.

When the cerebral blood flow augmenter contains a component other than isomaltulose, the content of isomaltulose may be appropriately set according to, e.g., the form and intended use of the cerebral blood flow augmenter. For example, the content of isomaltulose may be 10 mass % or more, 20 mass % or more, or 30 mass % or more, and may be 70 mass % or less, 60 mass % or less, or 50 mass % or less based on the total amount of the cerebral blood flow augmenter.

The cerebral blood flow augmenter can be used as a food composition or a pharmaceutical composition. The food composition or pharmaceutical composition according to the present embodiment may be provided in the form of, for example, a health food, a food for specified health uses, a functional food, a food with nutrient function claims, a supplement, a quasi-drug, or a pharmaceutical product. The food composition according to the present embodiment may have, for example, a label indicating that the cerebral blood flow is increased, the brain activity is promoted, or the vigilance is improved. In addition, the preparation may have any one of the forms such as a solid (e.g., powder, granules), a liquid (e.g., solution, suspension), or a paste, and may have any one of dosage forms such as a powder, a pill, a granule, a tablet, a capsule, a troche, a liquid, or a suspension.

The cerebral blood flow augmenter may be administered parenterally, but is preferably administered orally. As the dose, for example, the dose of isomaltulose per time may be 3 g or more, 5 g or more, 7 g or more, or 10 g or more, and may be 40 g or less, 30 g or less, 20 g or less, 15 g or less, 12 g or less, or 10 g or less. In addition, the dose of isomaltulose per day may be 3 g or more, 5 g or more, or 10 g or more, and may be 40 g or less, 30 g or less, or 20 g or less. The cerebral blood flow augmenter according to the present embodiment exhibits a desired effect even if the dose is smaller than the conventional one. Since isomaltulose is digested and absorbed more slowly than other carbohydrates, isomaltulose has a characteristic feature, that is, the level of glucose in blood rises slowly and remains higher than when fasting for a long time. Based on the characteristic feature, various functions are exerted. However, even if the dose of the cerebral blood flow augmenter according to the present embodiment is less likely to raise the blood glucose level, a function of increasing cerebral blood flow is exerted.

Since the cerebral blood flow augmenter according to the present embodiment also has an action of promoting brain activity and an action of improving the vigilance, the present invention can also be regarded as a brain activity promoter or a vigilance improver containing isomaltulose as an active ingredient. As a specific embodiment of the brain activity promoter or the vigilance improver, the embodiment described in the section of the cerebral blood flow augmenter described above is applicable.

As an embodiment of the present invention, there is a method for increasing cerebral blood flow in a subject, including a step of administering an effective amount of a cerebral blood flow augmenter containing isomaltulose as an active ingredient to the subject in need thereof, a method for promoting brain activity in a subject, including a step of administering an effective amount of a brain activity promoter containing isomaltulose as an active ingredient to the subject in need thereof, or a method for improving the vigilance of a subject, including a step of administering an effective amount of a vigilance improver containing isomaltulose as an active ingredient to the subject in need thereof.

The subject in each of the above methods may be a mammal, and is preferably a human. For example, the embodiment, administration method and dose (intake) of the cerebral blood flow augmenter, the brain activity promoter, or the vigilance improver in each of the above methods may be the same as those described above.

An embodiment of the present invention may include use of isomaltulose for producing a cerebral blood flow augmenter, use of isomaltulose for producing a brain activity promoter, or use of isomaltulose for producing a vigilance improver.

Embodiments of the cerebral blood flow augmenter, the brain activity promoter, and the vigilance improver in individual uses described above may be the same as those described above.

As an embodiment of the present invention, there may be isomaltulose for use in increasing cerebral blood flow, isomaltulose for use in promoting brain activity, or isomaltulose for use in improving vigilance.

### EXAMPLES

Hereinafter, the present invention will be described more specifically with reference to Examples. However, the present invention is not limited to these Examples.

As the subjects, 64 healthy men and women of 20 to 60 years old were employed. The effects of intake of isomaltulose on cerebral blood flow and brain activity were evaluated based on measurement of the response time to a cognitive task (Digit vigilance task (DVT)) and change of the cerebral blood flow in accordance with the following test. Test methods are more specifically shown below.

### Test Schedule

Each subject was tested in accordance with the schedule shown in Table 1. In the test, each block including the steps shown in Table 1 was performed in one hour, and the 1st block to the 5th block were continuously performed on the same day. As shown in Table 1, in this test, DVT (will be described in detail later) was performed 5 times in total. Before the start of the 2nd DVT, an aqueous solution prepared by dissolving 10.0 g of isomaltulose in 100 mL of water was given as a test food. After the completion of DVT and before the next DVT, the Uchida Kraepelin test was performed. The Uchida Kraepelin test is a test of adjacent single-digit addition. In this Example, the test was performed in order to apply a load such as fatigue to the subject in order to control the break time.

TABLE 1

| | Content of Implementation |
|---|---|
| 1st block | Water intake (100 mL) → 1st DVT, questionnaire (15 |
| (Start time: | minutes) → Uchida Kraepelin test, questionnaire (35 |
| 14:00) | minutes) → break (5 minutes) |
| 2nd block | Test food (isomaltulose 10.0 g) intake → 2nd DVT, |
| (Start time: | questionnaire (15 minutes) → Uchida Kraepelin test, |
| 15:00) | questionnaire (35 minutes) → break (5 minutes) |
| 3rd block | Water intake (100 mL) → 3rd DVT, questionnaire (15 |
| (Start time: | minutes) → Uchida Kraepelin test, questionnaire (35 |
| 16:00) | minutes) → break (5 minutes) |
| 4th block | Water intake (100 mL) →4th DVT, questionnaire (15 |
| (Start time: | minutes) → Uchida Kraepelin test, questionnaire (35 |
| 17:00) | minutes) → break (5 minutes) |
| 5th block | Water intake (100 mL) → 5th DVT, questionnaire (15 |
| (Start time: | minutes) → Uchida Kraepelin test, questionnaire (35 |
| 18:00) | minutes |

### DVT

DVT is a task mainly used for measuring the vigilance (Non Patent Literature 1 (Joel S. Warm et al., Vigilance requires hard mental work and is stressful, Human Factors, Vol. 50 (3), pp. 433-41, 2008)). DVT is also used for measuring, e.g., alertness, and mental fatigue (Non Patent Literature 2 (Watson, et al., The impact of blackcurrant juice on attention, mood and brain wave spectral activity in young healthy volunteers, Nutritional Neuroscience, Vol. 22 (8), pp. 596-606, 2019)). The method will be more specifically described below. In the state where a target number or character is always displayed on the right side of the screen, another number or character is displayed at the center of the screen. The subject is instructed to respond by operating the keyboard only when the number or character displayed at the center of the screen coincides with the number or character displayed on the right side of the screen. The time from display of the number or character to the subject's response is measured. In this test, after a predetermined number of tasks were continuously performed for 2 minutes, a 30-second break was interposed. This program was repeated 6 times (the time taken for the tasks was 15 minutes in total).

### Subjective Questionnaire Survey

First, “Last night, did you sleep better than usual?” was asked. After DVT or Uchida Kraepelin test, the survey for asking subjectivity of the subjects was conducted as to “How are you feeling right now?”, “Are you feeling tired right now?”, “Are you feeling irritated right now?”, “Are you feeling sleepy right now?”, and “Did you work diligently on the task until the end?”. The questionnaire survey was performed on the basis of the VAS (Visual Analogue Scale) method, which is a method of indicating a position relative to the evaluation words at both ends.

### Control Group

The subjects mentioned above were instructed to take a test diet containing glucose instead of isomaltulose on another day and the same test was performed (crossover test). They constituted a control group.

### Evaluation

### [Evaluation of Response Time in DVT]

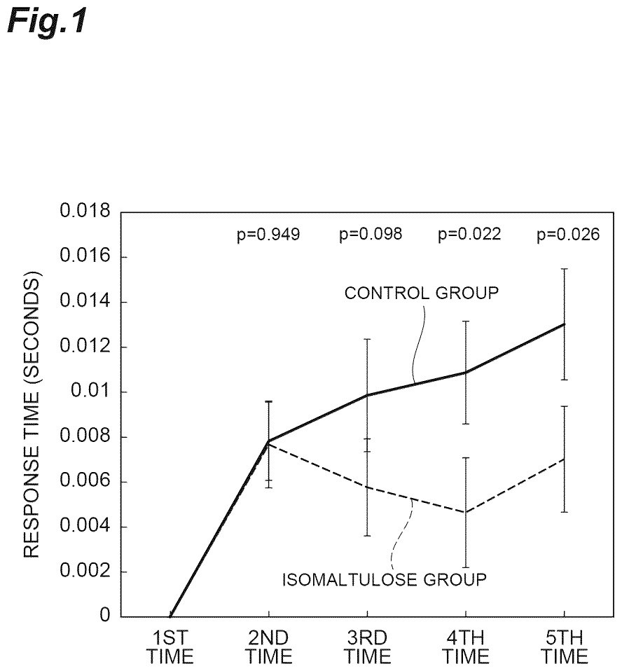

In DVT, the time from the display of a number or a character at the center of the screen to the response by each of the subjects based on the recognition that the number or the character coincided with the number or the character displayed on the right side of the screen, was measured and an average response time of all of the subjects was obtained. Specifically, the average response time of 6 tasks repeatedly performed for 15 minutes (task time) per subject was obtained, and then the average value of all of the subjects was further calculated. FIG. 1 illustrates how the average response time changes with an increase in the number of DVT times, on the basis that the average value of the response time at the 1st DVT (performed in the 1st block) regarded as 0. More specifically, in the graph of FIG. 1, a larger value of the response time on the vertical axis means that a larger response time (the response time increases) is required than that of the 1st DVT.

As shown in FIG. 1, it is apparent that the response time in DVT is gradually increases in the control group taking glucose, whereas the response time is less likely to increase in the group taking isomaltulose, compared to the control group even when DVT is performed a plurality of times. In other words, it can be said that a state of handing a task at a high speed can be maintained for a longer time, by intake of isomaltulose and a state of high vigilance can be maintained for a longer time. In addition, since DVT is also a task for measuring, e.g., alertness, and mental fatigue, it can be said that a high-alertness state can be maintained for a long time and mental fatigue can be suppressed by intake of isomaltulose.

### Evaluation of Cerebral Blood Flow

Using a brain activity measuring apparatus (HOT-2000, manufactured by NeU Corporation) based on fNIRS (near-Infrared spectroscopy), a change in cerebral blood flow in the dorsal lateral prefrontal area was measured during DVT. According to this apparatus, a change in hemoglobin concentration in blood can be detected by irradiation with near-infrared light, with the result that a change in cerebral blood flow in an active area of the brain can be measured.

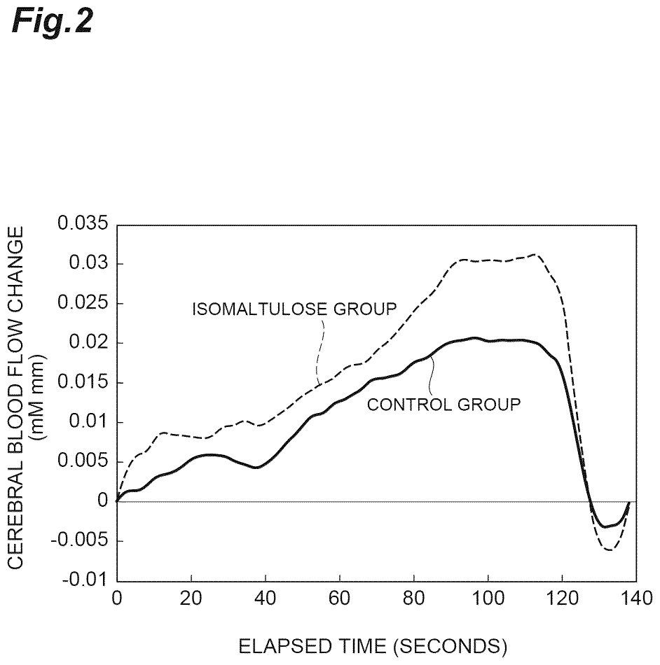

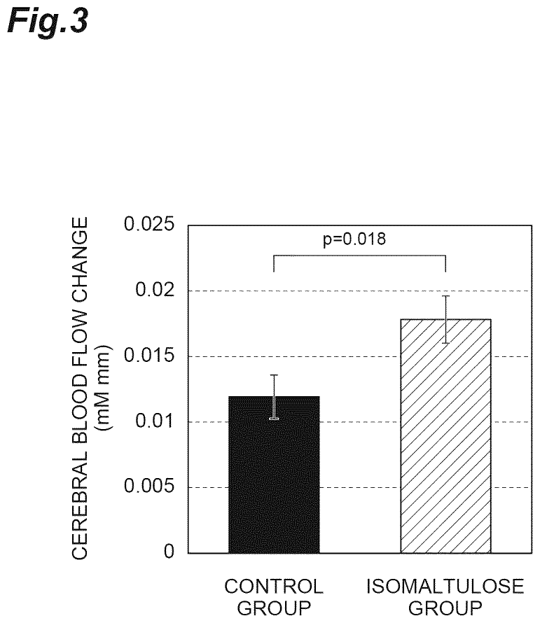

FIG. 2 is a graph showing the transition of an average value of a cerebral blood flow change over time, with the cerebral blood flow at the start of DVT set to zero, in a total of 5 (1st to 5th blocks) DVT tests. FIG. 3 shows an average value of the cerebral blood flow change from 0 to 120 seconds, based on the transition of the average value of cerebral blood flow change shown in FIG. 2. As shown in FIGS. 2 and 3, in the isomaltulose intake group, the change in cerebral blood flow was significantly large throughout 5 times of DVT as compared to the glucose intake group (control group). Thus, it is found that in the case where isomaltulose is taken, the cerebral blood flow increases as compared to the case where glucose is taken, with the result that brain activity is more activated.

### [Result of Questionnaire Survey to Subjects]

A questionnaire survey was conducted after the task. As a result, to the question “have you dealt with the task diligently to the end?” in the questionnaire survey conducted at the 3rd to 5th block, the isomaltulose intake group answered that “dealt with the task diligently” rather than the control group. Thus, it can be said that motivation to work could be maintained for a long time by intake of isomaltulose.

1. A cerebral blood flow augmenter containing isomaltulose as an active ingredient.

2. A brain activity promoter containing isomaltulose as an active ingredient.

3. A vigilance improver containing isomaltulose as an active ingredient.
